(19) United States
(12) Patent Application Publication
(10) Pub. No.: US 2022/0249711 A1
(43) Pub. Date: Aug. 11, 2022

(54) METHODS FOR TREATING TUMORS

(71) Applicants: CENTRE HOSPITALIER UNIVERSITAIRE DE GRENOBLE, La Tronche (FR); UNIVERSITE GRENOBLE ALPES, Saint Martin D'heres (FR); NH THERAGUIX, Meylan (FR)

(72) Inventors: Olivier TILLEMENT, Fontaines Saint Martin (FR); François LUX, Lyon (FR); Sandrine DUFORT, Meylan (FR); Camille VERRY, Saint-Ismier (FR); Géraldine LEDUC, Crolles (FR)

(21) Appl. No.: 17/597,903

(22) PCT Filed: Jul. 28, 2020
(86) PCT No.: PCT/EP2020/071286

§ 371 Date: Jan. 28, 2022

(30) Foreign Application Priority Data
Jul. 29, 2019 (IB) PCT/IB2019/000880

(51) Int. Cl. A61K 51/06; A61K 9/00; A61K 51/12; A61K 49/12; A61K 49/18; A61P 35/00

(52) U.S. Cl. CPC A61K 51/065; A61K 9/0019; A61P 35/00; A61K 49/12; A61K 49/1881; A61K 51/1244

(57) ABSTRACT

The disclosure relates to methods for treating tumors. In particular, the disclosure relates to a method of treating a tumor by ionizing radiations in a subject in need thereof, said method comprising the steps of:
(i) injecting a first therapeutically effective amount of high-Z element containing nanoparticles as radiosensitizing agents in said subject in need thereof within a period between 2 and 7 days prior to the first irradiation of the tumor,
(ii) injecting a second therapeutically effective amount of the same or different high-Z element containing nanoparticles within a period between 1 hour to 12 hours prior to the first irradiation of the tumor, and,
(iii) irradiating the tumor of said subject with a therapeutically efficient dose of radiations;
   wherein said high-Z element containing nanoparticles are nanoparticles containing an element with an atomic Z number higher than 40 and said nanoparticles have a mean hydrodynamic diameter below 10 nm.

### TECHNICAL DOMAIN

The disclosure relates to methods for treating tumors. In particular, the disclosure relates to a method of treating a tumor by ionizing radiations in a subject in need thereof, said method comprising the steps of:
(i) injecting a first therapeutically effective amount of high-Z element containing nanoparticles as radiosensitizing agents in a subject in need thereof within a period between 2 and 10 days prior to the first irradiation of the tumor,
(ii) injecting a second therapeutically effective amount of the same or different high-Z element containing nanoparticles within a period between 1 hour to 12 hours prior to the first irradiation of the tumor, and,
(iii) irradiating the tumor of said subject with a therapeutically efficient dose of radiations;
   wherein said high-Z element containing nanoparticles are nanoparticles containing an element with an atomic Z number higher than 40, and said nanoparticles have a mean hydrodynamic diameter below 10 nm.

### BACKGROUND

The radiation therapy (also known as radiotherapy) is one of the most used anti-tumor strategies. More than half of all patients with cancer are treated with ionizing radiation (IR) alone or in combination with surgery or chemotherapy.′ Recent progresses realized in medical physics (with the development of low/high energy radiation, the implementation of mono-, hypo- or hyper-fractionation schedule and the diversification of dose rates used) and the development of innovative medical technologies (such as the 3D-conformational radiotherapy (3D-CRT), the intensity modulated radiation therapy (IMRT), the stereotactic radiosurgery (SRS) and the functional imaging)) contribute to better deliver the efficient doses of radiation on tumors whilst sparing surrounding healthy tissues, which is the most usual side effect of radiation therapy.² Several applications of nanomedicine (such as radioisotope-labeled or metallic nanoparticles) have been developed to improve this therapeutic index by using nanomaterials as imaging or contrast agents to better deliver the radiation doses into tumor sites and/or as radiosensitizers, to enhance the dose deposition in tumors and reduce irradiation-related side-effects.^{3, 4, 5, 6, 7} Considering that the radiation dose absorbed by any tissues is related to the square of relative atomic number (Z²) of the material (where Z is the atomic number)⁸, nanoparticles containing high-Z atoms (such as gold or gadolinium) have been extensively investigated for their potential to improve radiotherapy. Under exposure to ionizing radiations, heavy-metal based nanoparticles produce photons and Auger electrons that improve the total dose rate deposition into the tumors, induce the production of reactive oxygen species (ROS) and cause cellular damages on many tumors (including for example melanoma, glioblastoma, breast and lung carcinomas).^{9, 10, 11} Despite the fact that several preclinical animal models revealed the ability of the combination of heavy-metal based nanoparticles with ionizing radiation to reduce tumor growth, there is still a need to improve the use of radiosensitizing nanoparticles in anti-cancer treatments in combination with ionizing radiations.

In particular, an important rule for the success of a therapeutic strategy involving radiosensitizing nanoparticles rests on the preservation of the surrounding healthy tissue while optimizing the concentration and distribution of the radiosensitizing nanoparticles in the tumors to be treated.

In the present disclosure, the inventors now surprisingly found that more than 10% of the Gd-based nanoparticles initially targeted to the tumors were still visible in the tumors 8 days after the first injection. This unexpectedly long persistence of the nanoparticles in the human tumors enable the inventor to design a new regimen of administration of the nanoparticles for use as radiosensitizing agents in combination with radiation therapy in a human patient. The method of the present disclosure comprises at least two injections of the nanoparticles prior to irradiation with one or more injections being classically done less than 24 hours prior to irradiation, but further comprising a first injection being done within a period between 2 and 10 days prior to the first irradiation of the tumor.

### BRIEF DESCRIPTION

Accordingly, the present disclosure relates to a method of treating a tumor by ionizing radiations in a subject in need thereof, the method comprising:
(i) injecting a first therapeutically effective amount of high-Z element containing nanoparticles as radiosensitizing agents in said subject in need thereof within a period between 2 and 10 days prior to the first irradiation of the tumor, preferably between 2 and 7 days,
(ii) injecting a second therapeutically effective amount of the same or different high-Z element containing nanoparticles within a period between 1 hour to 12 hours prior to the first irradiation of the tumor, and,
(iii) irradiating the tumor of said subject with a therapeutically efficient dose of radiations; wherein said high-Z element containing nanoparticles are nanoparticles containing an element with an atomic Z number higher than 40, preferably higher than 50, and said nanoparticles have a mean hydrodynamic diameter below 10 nm, preferably below 6 nm, for example between 2 and 6 nm.

In specific embodiments of the methods, said nanoparticles are injected intravenously.

In specific embodiments, said nanoparticles comprise, as high-Z element, a rare earth metal, or a mixture of rare earth metals. In more specific embodiments, said nanoparticles comprise, as high-Z element, gadolinium, bismuth, or a mixture thereof.

Typically, said nanoparticles comprise chelates of high-Z element, for example chelates of rare earth elements.

In specific embodiments, the nanoparticles for use in the above methods comprise
(i) polyorganosiloxane,
(ii) chelates covalently bound to said polyorganosiloxane; and,
(iii) high-Z elements complexed by the chelates.

In specific embodiments, said nanoparticles for use in the above methods comprise
(i) polyorganosiloxanes with a silicon weight ratio of at least 8% of the total weight of the nanoparticle, preferably between 8% and 50%,
(ii) chelates covalently bound to said polyorganosiloxane, in a proportion comprising between 5 and 100, preferably between 5 and 20 per nanoparticle, and,
(iii) high-Z elements complexed to the chelates.

In specific embodiments, said nanoparticles for use of in the above methods may comprise chelates for complexing the high-Z elements, obtained by grafting one or more of the following cheating agents on said nanoparticles: DOTA, DTPA, EDTA, EGTA, BAPTA, NOTA, DOTAGA, and DTPABA, or their mixtures.
In a particularly preferred embodiment, said nanoparticles are gadolinium-chelated polysiloxane nanoparticules of the following formula

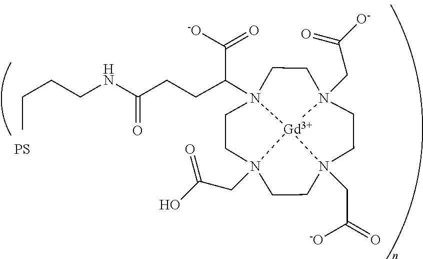

wherein PS is a matrix of polysiloxane, and,
n is comprised between 5 and 50, preferably 5 and 20, and wherein the hydrodynamic diameter is comprised between 2 and 6 nm.

In specific embodiments, said nanoparticles are AGuIX nanoparticles.

In specific embodiments, said nanoparticles are a lyophilized powder contained in a pre-filled vial to be reconstituted in an aqueous solution for intravenous injection.

In specific embodiments, the nanoparticles for use in the above methods are comprised in an injectable solution at a concentration between 50 and 150 mg/mL, and preferably between 80 and 120 mg/mL, for example 100 mg/mL.

Typically, a therapeutically effective amount administered at each injecting step may be comprised between 50 mg/kg and 150 mg/kg, typically, between 80 and 120 mg/kg, for example 100 mg/kg.

In specific embodiments, said tumor to be treated is a solid tumor, preferably selected from the group consisting of glioblastoma, brain metastases, meniningioma, or primary tumor of uterine cervix, rectum, lung, head and neck, prostate, colorectal, liver, and pancreas cancers. Typically, said tumor is a brain metastase, typically a brain metastase from melanoma, lung, breast, kidney, colon primary cancers.

In specific embodiments, said subject is exposed at step (iii) to a whole brain radiation therapy. For example, said whole brain radiation therapy consists of exposing the subject to a total dose of ionizing radiations between 25 and 35 Gy, for example 30 Gy. Typically, the subject is exposed to a dose of ionizing radiations per fraction of about 3 Gy, and the total dose is administered preferably in a maximum of 10 fractions.

In specific embodiments, the method comprises a step of injecting a third therapeutically effective amount of the same or different high-Z element containing nanoparticles within 5-10 days after the second injecting step, for example 7 days after the second injecting step.

In specific embodiments, the method further comprises a step of imaging the tumor by magnetic resonance imaging (MRI) after the first injecting step of said nanoparticles, wherein said nanoparticles is used as a T1 contrast agent for said MRI.

### LEGENDS TO FIGURES

FIG. 1. MRI enhancement versus administered dose. Each point on the graph corresponds to an MRI enhancement value measured in a metastasis with longest diameter larger than 1 cm. MRI enhancements were found statistically different between each dose, the pooled 15-30 mg/kg dose, the pooled 50-75 mg/kg and 100 mg/kg.

FIG. 2. MRI enhancement versus AGuIX concentration. Each point on the graph corresponds to an MRI enhancement and AGuIX concentration value measured in a metastasis with longest diameter larger than 1 cm of patient #13. The black curve corresponds to a linear regression applied to the series of points. Dashed curved correspond to the 95% confidence bands.

FIG. 3. MRI enhancement one week after the administration of nanoparticles. Part of the signal enhancement map (color coded) of patient #13 is superimposed to the native 3D T₁-weighted images obtained 2 hours post i.v. injection (left side image) to the patient and one week later (right side image). The arrows are pointing the AGuIX-enhanced metastasis.

FIG. 4. Synopsis of the phase II clinical study and objectives

### DETAILED DESCRIPTION

The present disclosure follows in part from the surprising findings as shown by the inventors of a long persistence in human tumors of certain nanoparticles for use as radiosensitizing agents in cancer radiation therapies.

The advantageous effects of the methods of treatment of the present disclosure are linked in particular to two features of the nanoparticles:
(i) they contain high-Z elements, typically complexes of high-Z cations with radiosensitizing properties;
(ii) they have a small mean hydrodynamic diameter.

As used herein, the term “radiosensitizing” would be readily understood by one of ordinary skill in the art and generally refers to the process of increasing the sensitivity of the cancer cells to radiation therapy (e.g., photon radiation, electron radiation, proton radiation, heavy ion radiation, and the like).

Said high-Z element as used herein is an element with an atomic Z number higher than 40, for example higher than 50.

In specific embodiments, said high-Z element is selected among the heavy metals, and more preferably, Au, Ag, Pt, Pd, Sn, Ta, Zr, Tb, Tm, Ce, Dy, Er, Eu, La, Nd, Pr, Lu, Yb, Bi, Hf, Ho, Pm, Sm, In, and Gd, and mixtures thereof.

The high-Z elements are preferably cationic elements, either comprised in the nanoparticles as oxide and/or chalcogenide or halide or as complexes with chelating agents, such as organic chelating agents.

The size distribution of the nanoparticles is, for example, measured using a commercial particle sizer, such as a Malvern Zêtasizer Nano-S particle sizer based on PCS (Photon Correlation Spectroscopy).

For the purposes of the invention, the term “mean hydrodynamic diameter” or “mean diameter” is intended to mean the harmonic mean of the diameters of the particles. A method for measuring this parameter is also described in standard ISO 13321:1996.

Nanoparticles with a mean hydrodynamic diameter for example between 1 and 10 nm, and even more preferably between 1 and 8 nm or for example between 2 and 6 nm, or typically around 3 nm, are suitable for the methods disclosed herein. In particular, they have been shown to provide excellent passive targeting in tumors, including brain tumors, after intravenous injection, and a rapid renal elimination (and therefore low toxicity).

In another embodiment, which may be combined with previous embodiments, the nanoparticles can be also advantageously used as an imaging or a contrast agent, for example, in image-guided radiation therapy.

As used herein, the term “contrast agent” is intended to mean any product or composition used in medical imaging for the purpose of artificially increasing the contrast making it possible to visualize a particular anatomical structure (for example certain tissues or organs) or pathological anatomical structures (for example tumors) with respect to neighboring or non-pathological structures. The term “imaging agent” is intended to mean any product or composition used in medical imaging for the purpose of creating a signal making it possible to visualize a particular anatomical structure (for example certain tissues or organs) or pathological anatomical structures (for example tumors) with respect to neighboring or non-pathological structures. The principle of how the contrast or imaging agent operates depends on the imaging technique used.

In some embodiments, the imaging is performed using magnetic resonance imaging (MRI), computed tomography imaging, positron emission tomography imaging, or any combination thereof.

Advantageously, it will be possible to combine the use of the nanoparticles in the method of treatment of the disclosure, and for an in vivo detection of tumors by MRI, enabling, for example, monitoring of the therapeutic treatment as described in the present disclosure.

Preferably, only lanthanides, including at least 50% by weight of gadolinium (Gd), of dysprosium (Dy), of lutetium (Lu), for bismuth (Bi) or of holmium (Ho), or mixtures thereof, (relative to the total weight of high-Z elements in the nanoparticles), for example at least 50% by weight of gadolinium, will be chosen as high-Z elements in the nanoparticles.

In a particularly preferred embodiment, said nanoparticle for use in the method of the present disclosure is a gadolinium-based nanoparticle.

In specific embodiments, said high-Z elements are cationic elements complexed with organic chelating agents, for example selected from chelating agents with carboxylic acid, amine, thiol, or phosphonate groups.

In preferred embodiment, the nanoparticles further comprise a biocompatible coating in addition to the high-Z element, and, optionally, the chelating agents. Agent suitable for such biocompatible includes without limitation biocompatible polymers, such as polyethylene glycol, polyethyleneoxide, polyacrylamide, biopolymers, polysaccharides, or polysiloxane.

In particular embodiments, the nanoparticles are chosen such that they have a relaxivity r1 per particle of between 50 and 5000 mM⁻¹.s⁻¹ (at 37° C. and 1.4 T) and/or a Gd weight ratio of at least 5%, for example between 5% and 30%.

In one specific embodiment, said nanoparticles with a very small hydrodynamic diameter, for example between 1 and 10 nm, preferably between 2 and 6 nm, are nanoparticles comprising chelates of high-Z elements, for example chelates of rare earth elements. In certain embodiments, said nanoparticles comprise chelates of gadolinium or bismuth.

In specific embodiments which may be combined with any of the previous embodiments, said high-Z element containing nanoparticles comprise
polyorganosiloxane,
chelating agents covalently bound to said polyorganosiloxane,
high-Z elements complexed by the chelating agents.

As used herein, the term “chelating agent” refers to a group capable of complexing one or more metal ions.

Exemplary chelating agents include, but not limited to, 1,4,7-triazacyclononanetriacetic acid (NOTA), 1,4,7,10-tetraazacyclododecane-1,4,7,10-tetraacetic acid (DOTA), 1,4,7-triazacyclononane-I-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-0,0′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine tnacetic acid (HEDTA), 1,4,8,11-tetraazacyclotetradecane-N,N′,N″,N′″-tetraacetic acid (TETA), and 1, 4,7,10-tetraaza-1,4,7,10-tetra-(2-carbamoyl methyl)-cyclododecane (TCMC) and 1,4,7,10-tetraazacyclododececane, 1-(glutaric acid)-4,7,10-triacetic acid (DOTAGA).

In some embodiments, said chelating agent is selected among the following:

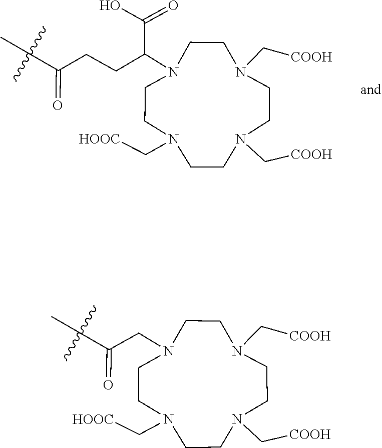 [SMILES: *C(=O)CCC(C(=O)O)N1CCN(CC(=O)O)CCN(CC(=O)O)CCN(CC(=O)O)CC1 ; *C(=O)CN1CCN(CC(=O)O)CCN(CC(=O)O)CCN(CC(=O)O)CC1]

wherein the wavy bond indicates the bond connecting the chelating agent to a linking group of a biocompatible coating forming the nanoparticle.

In a specific embodiment, that may be preferably combined with the previous embodiment, said chelates of rare earth element are chelates of gadolinium and/or bismuth, preferably DOTA or DOTAGA chelating Gd³⁺ and/or Bi.

In specific and preferred embodiments, the ratio of high-Z element per nanoparticle, for example the ratio of rare earth elements, e.g. gadolinium (optionally as chelated with DOTAGA) per nanoparticle, is between 3 and 100, preferably between 5 and 20, typically around 10.

For imaging by scintigraphy, the nanoparticles may additionally comprise a radioactive isotope that can be used in scintigraphy, and that is preferably chosen from the group consisting of the radioactive isotopes of In, Tc, Ga, Cu, Zr, Y or Lu, for example: ¹¹¹In, ^{99m}Tc, ⁶⁷Ga, ⁶⁸Ga, ⁶⁴Cu, ⁸⁹Zr, ⁹⁰Y or ¹⁷⁷Lu.

For fluorescence in the near-infrared range, the nanoparticles may additionally comprise a lanthanide chosen from Nd, Yb or Er.

For fluorescence in the visible range, the nanoparticles may additionally comprise a lanthanide chosen from Eu or Tb can be used.

For fluorescence in the near-infrared range, the nanoparticles may additionally comprise an organic fluorophore chosen from Cyanine 5.5, Cyanine 7, Alexa 680, Alexa 700, Alexa 750, Alexa 790, Bodipy, ICG.

In specific embodiments, the hybrid nanoparticles are of core-shell type. Nanoparticles of core-shell type, based on a core consisting of a rare earth oxide and of an optionally functionalized polyorganosiloxane matrix are known (see in particular WO 2005/088314, WO 2009/053644).

The nanoparticles may further be functionalized with molecules which allow targeting of the nanoparticles to specific tissues. Said agents can be coupled to the nanoparticle by covalent couplings, or trapped by non-covalent bonding, for example by encapsulation or hydrophilic/hydrophobic interaction or using a chelating agent.

In one specific embodiment, use is made of hybrid nanoparticles comprising:
a polyorganosiloxane (POS) matrix including, rare earth cations Mⁿ⁺, n being an integer between 2 and 4, optionally partly in the form of a metal oxide and/or oxyhydroxide, optionally associated with doping cations D^{m+}, m being an integer between 2 and 6, D preferably being a rare earth metal other than M, an actinide and/or a transition element;
a chelate covalently bound to the POS via a covalent bond —Si—C—,
the Mⁿ⁺ cations and, where appropriate, D^{m+} cations being complexed by the chelates.

In the case of a structure of core-shell type, the POS matrix forms the superficial layer surrounding the metal cation-based core. Its thickness can range from 0.5 to 10 nm, and can represent from 25% to 75% of the total volume.

The POS matrix acts as protection for the core with respect to the external medium (in particular protection against hydrolysis) and it optimizes the properties of the contrast agents (luminescence, for example). It also allows the functionalization of the nanoparticle, via the grafting of chelating agents and of targeting molecules.

Ultrafine Nanoparticles

In a specific embodiment, said nanoparticles are gadolinium-chelated polysiloxane nanoparticles of the following formula

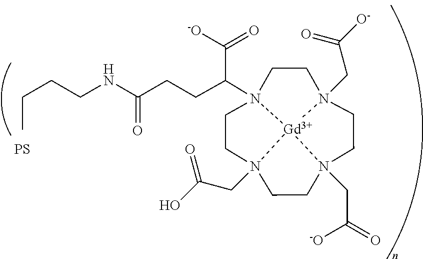

wherein PS is a matrix of polysiloxane, and wherein n is comprised between 5 and 50, typically 5 and 20, and wherein the hydrodynamic diameter is comprised between 2 and 6 nm.

More specifically, said gadolinium-chelated polysiloxane nanoparticle as described in the above formula is an AGuIX ultrafine nanoparticles as described in the next section.

Such ultrafine nanoparticles that can be used according to the methods of the disclosure may be obtained or obtainable by a top-down synthesis route comprising the steps of:
a. obtaining a metal (M) oxide core, wherein M is a high-Z element as described previously, preferably gadolinium,
b. adding a polysiloxane shell around the M oxide core, for example via a sol gel process,
c. grafting a chelating agent to the POS shell, so that the chelating agent is bound to said POS shell by an —Si—C— covalent bond, thereby obtaining a core-shell precursor nanoparticle, and,
d. purifying and transferring the core-shell precursor nanoparticle in an aqueous solution,

wherein the grafted agent is in sufficient amount to dissolve the metal (M) oxide core at step d. and to complex the cationic form of (M) thereby reducing the mean hydrodynamic diameter of the resulting hybrid nanoparticle to a mean diameter less than 10 nm, for example, between 1 and 8 nm, typically less than 6 nm, for example between 2 and 6 nm.

These nanoparticles obtained according to the mode described above do not comprise a core of metal oxide encapsulated by at least one coating. More details regarding the synthesis of these nanoparticles are given hereafter.

This top-down synthesis method results in observed sizes typically of between 1 and 8 nm, more specifically between 2 and 6 nm. The term then used herein is ultrafine nanoparticles.

Alternatively, another “one-pot” synthesis method is described hereafter to prepare said core-free nanoparticles with a mean diameter less than 10 nm, for example, between 1 and 8 nm, typically between 2 and 6 nm.

Further details regarding these ultrafine or core-free nanoparticles, the processes for synthesizing them and their uses are described in patent application WO2011/135101, WO2018/224684 or WO2019/008040, which is incorporated by way of reference.

Process for Obtaining Preferred Embodiments of Nanoparticles for Use According to the Disclosure

Generally, those skilled in the art will be able to easily produce nanoparticles used according to the invention. More specifically, the following elements will be noted:

For nanoparticles of core-shell type, based on a core of lanthanide oxide or oxyhydroxide, use may be made of a production process using an alcohol as solvent, as described for example in P. Perriat et al., J. Coll. Int. Sci, 2004, 273, 191; O. Tillement et al., J. Am. Chem. Soc., 2007, 129, 5076 and P. Perriat et al., J. Phys. Chem. C, 2009, 113, 4038.

For the POS matrix, several techniques can be used, derived from those initiated by Stoeber (Stoeber, W; J. Colloid Interf Sci 1968, 26, 62). Use may also be made of the process used for coating as described in Louis et al. (Louis et al., 2005, Chemistry of Materials, 17, 1673-1682) or international application WO 2005/088314.

In practice, synthesis of ultrafine nanoparticles is for example described in Mignot et al. Chem. Eur. J. 2013, 19, 6122-6136: Typically, a precursor nanoparticle of core/shell type is formed with a lanthanide oxide core (via the modified polyol route) and a polysiloxane shell (via sol/gel); this object has, for example, a hydrodynamic diameter of around 5-10 nm. A lanthanide oxide core of very small size (adjustable less than 10 nm) can thus be produced in an alcohol by means of one of the processes described in the following publications: P. Perriat et al., J. Coll. Int. Sci, 2004, 273, 191; O. Tillement et al., J. Am. Chem. Soc., 2007, 129, 5076 and P. Perriat et al., J. Phys. Chem. C, 2009, 113, 4038.

These cores can be coated with a layer of polysiloxane according to, for example, a protocol described in the following publications: C. Louis et al., Chem. Mat., 2005, 17, 1673 and O. Tillement et al., J. Am. Chem. Soc., 2007, 129, 5076.

Chelating agents specific for the intended metal cations (for example DOTAGA for Gd³+) are grafted to the surface of the polysiloxane; it is also possible to insert a part thereof inside the layer, but the control of the formation of the polysiloxane is complex and simple external grafting gives, at these very small sizes, a sufficient proportion of grafting.

The nanoparticles may be separated from the synthesis residues by means of a method of dialysis or of tangential filtration, for example on a membrane comprising pores of appropriate size.

The core is destroyed by dissolution (for example by modifying the pH or by introducing complexing molecules into the solution). This destruction of the core then allows a scattering of the polysiloxane layer (according to a mechanism of slow corrosion or collapse), which makes it possible to finally obtain a polysiloxane object with a complex morphology, the characteristic dimensions of which are of the order of magnitude of the thickness of the polysiloxane layer, i.e. much smaller than the objects produced up until now.

Removing the core thus makes it possible to decrease from a particle size of approximately 5-10 nanometers in diameter to a size below 8 nm, for example between 2-6 nm. Furthermore, this operation makes it possible to increase the number of M (e.g. gadolinium) per nm³ in comparison with a theoretical polysiloxane nanoparticle of the same size but comprising M (e.g. gadolinium) only at the surface. The number of M for a nanoparticle size can be evaluated by virtue of the M/Si atomic ratio measured by EDX. Typically, this number of M per ultrafine nanoparticle may be comprised between 5 and 50.

In one specific embodiment, the nanoparticle according to the disclosure comprises a chelating agent which has an acid function, for example DOTA or DOTAGA. The acid function of the nanoparticle is activated for example using EDC/NHS (1-ethyl-3-(3-dimethylaminopropyl)carbodiimide/N-hydrosuccinimide) in the presence of an appropriate amount of targeting molecules. The nanoparticles thus grafted are then purified, for example by tangential filtration.

Alternatively, the nanoparticles according to the present disclosure may be obtained or obtainable by a synthesis method (“one-pot synthesis method”) comprising the mixing of at least one hydroxysilane or alkoxysilane which is negatively charged at physiological pH and at least one chelating agent chosen from polyamino polycarboxylic acids with
at least one hydroxysilane or alkoxysilane which is neutral at physiological pH, and/or
at least one hydroxysilane or alkoxysilane which is positively charged at physiological pH and comprises an amino function,
   wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

According to a more specific embodiment of such one pot synthesis method, the method comprises the mixing of at least one alkoxysilane which is negatively charged at physiological pH, said alkoxysilane being chosen among APTES-DOTAGA, TANED, CEST and mixtures thereof, with
at least alkoxysilane which is neutral at physiological pH, said alkoxysilane being chosen among TMOS, TEOS and mixtures thereof, and/or
APTES which is positively charged at physiological pH,
wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

According to a specific embodiment, the one pot synthesis method comprises the mixing of APTES-DOTAGA which is negatively charged at physiological pH with
at least one alkoxysilane which is neutral at physiological pH, said alkoxysilane being chosen among TMOS, TEOS and mixtures thereof, and/or
APTES which is positively charged at physiological pH,
wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

AGuIX Nanoparticles

In a more specific embodiment, said gadolinium-chelated polysiloxane based nanoparticle is the core-free ultrafine AGuIX nanoparticle of the formula below

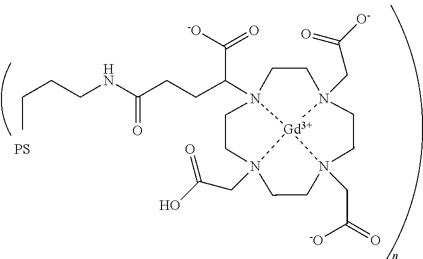

wherein PS is polysiloxane and n is, on average, about 10, and having a hydrodynamic diameter of 4±2 nm and a mass of about 10±1 kDa.

Said AGuIX nanoparticle can also be described by the average chemical formula:

(GdSi₄₋₇C₂₄₋₃₀N₅₋₈O₁₅₋₂₅H₄₀₋₆₀,5-10H₂O)ₓ

Pharmaceutical Formulations of the Nanoparticles for Use According to the Disclosed Methods

When employed as pharmaceuticals, the compositions comprising said high-Z nanoparticles for use as provided herein can be administered in the form of pharmaceutical formulation of a suspension of nanoparticles. These formulations can be prepared as described herein or elsewhere, and can be administered by a variety of routes, depending upon whether local or systemic treatment is desired and upon the area to be treated.

In particular, said pharmaceutical formulations for use as described herein, contain, as the active ingredient, a suspension of high-Z containing nanoparticles, as provided herein, in combination with one or more pharmaceutically acceptable carriers (excipients). In making a pharmaceutical formulation provided herein, the nanoparticle composition may be, for example, mixed with an excipient or diluted by an excipient. When the excipient serves as a diluent, it can be a solid, semi-solid, or liquid material, which acts as a vehicle, carrier, or medium for the nanoparticle composition.

Thus, the pharmaceutical formulations can be in the form of powders, lozenges, elixirs, suspensions, emulsions, solutions, syrups, aerosols (as a solid or in a liquid medium), sterile injectable solutions, sterile packaged powders, and the like.

In specific embodiments, said pharmaceutical formulation for use as described herein, is sterile lyophilized powder, contained in a pre-filled vial to be reconstituted, for example in an aqueous solution for intravenous injection. In specific embodiments, said lyophilized powder comprises, as the active ingredient, an efficient amount of said high-Z containing nanoparticles, typically gadolinium-chelated polysiloxane based nanoparticles, and more specifically AGuIX nanoparticles as described herein. In certain specific embodiments, said lyophilized powder contains either about between 200 mg and 15 g per vial, for example between 280 and 320 mg of AGuIX per vial, typically 300 mg of AGuIX per vial or about between 800 mg and 1200 mg, for example 1 g of AGuIX per vial.

Such powder may further contain one or more additional excipients, and in particular CaCl₂), for example between 0.5 and 0.80 mg of CaCl₂), typically 0.66 mg of CaCl₂).

Said lyophilized powder may be reconstituted in an aqueous solution, typically water for injection. Accordingly, in specific embodiments, said pharmaceutical solution for use according to the present disclosure is a solution for injection, comprising, as the active ingredient, an efficient amount of said high-Z containing nanoparticles, typically gadolinium-chelated polysiloxane based nanoparticles, and more specifically AGuIX nanoparticles as described herein.

For example, said solution for injection for use in the methods as disclosed herein is a solution of gadolinium-chelated polysiloxane based nanoparticles, typically AGuIX nanoparticles, between 50 and 150 mg/mL, for example 80 and 120 mg/mL, typically 100 mg/mL, optionally comprising one or more additional pharmaceutically acceptable excipient, for example between 0.1 and 0.3 mg/mL of CaCl₂), typically 0.22 mg/mL of CaCl₂).

Methods of Use of the Present Disclosure

The present invention relates to a method of treating a tumor in a subject in need thereof, the method comprising
(i) injecting a first therapeutically effective amount of high-Z element containing nanoparticles as radiosensitizing agents in said subject in need thereof within a period between 2 and 10 days, preferably between 2 and 7 days, prior to the first irradiation of the tumor,
(ii) injecting a second therapeutically effective amount of the same or different high-Z element containing nanoparticles within a period between 1 hour to 12 hours prior to the first irradiation of the tumor, and,
(iii) irradiating the tumor of said subject with a therapeutically efficient dose of radiations; wherein said high-Z element containing nanoparticles are nanoparticles containing an element with an atomic Z number higher than 40, preferably higher than 50, and said nanoparticles have a mean hydrodynamic diameter below 10 nm, preferably below 6 nm, for example between 2 and 6 nm.

The disclosure also relates to high-Z element containing nanoparticles for use in a method of treating a tumor in a subject in need thereof, the method comprising
(i) injecting a first therapeutically effective amount of high-Z element containing nanoparticles as radiosensitizing agents in said subject in need thereof within a period between 2 and 10 days, preferably between 2 and 7 days, prior to the first irradiation of the tumor,
(ii) injecting a second therapeutically effective amount of the same or different high-Z element containing nanoparticles within a period between 1 hour to 12 hours prior to the first irradiation of the tumor, and,
(iii) irradiating the tumor of said subject with a therapeutically efficient dose of radiations; wherein said high-Z element containing nanoparticles are nanoparticles containing an element with an atomic Z number higher than 40, preferably higher than 50, and said nanoparticles have a mean hydrodynamic diameter below 10 nm, preferably below 6 nm, for example between 2 and 6 nm.

The disclosure further relates to high-Z element containing nanoparticles for use in the manufacture of a medicament for the treatment of a tumor in a subject in need thereof, the method comprising
(i) injecting a first therapeutically effective amount of high-Z element containing nanoparticles as radiosensitizing agents in said subject in need thereof within a period between 2 and 10 days, preferably between 2 and 7 days, prior to the first irradiation of the tumor,
(ii) injecting a second therapeutically effective amount of the same or different high-Z element containing nanoparticles within a period between 1 hour to 12 hours prior to the first irradiation of the tumor, and,
(iii) irradiating the tumor of said subject with a therapeutically efficient dose of radiations; wherein said high-Z element containing nanoparticles are nanoparticles containing an element with an atomic Z number higher than 40, preferably higher than 50, and said nanoparticles have a mean hydrodynamic diameter below 10 nm, preferably below 6 nm, for example between 2 and 6 nm.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of a tumor, the term “treatment” may refer to the inhibition of the growth of the tumor, or the reduction of the size of the tumor.

The term “patient” and “subject” which are used herein interchangeably refer to any member of the animal kingdom, including mammals and invertebrates. For example, mice, rats, other rodents, rabbits, dogs, cats, swine, cattle, sheep, horses, primates, fish, and humans. Preferably, the subject is a mammal, or a human being, including for example a subject that has a tumor.

In specific embodiments, said tumor is a solid tumor, preferably selected from the group consisting of glioblastoma, brain metastases, meniningioma, or primary tumor of uterine cervix, rectum, lung, head and neck, prostate, colorectal, liver, and pancreas cancers. In particular, said tumor is a brain metastase, typically a brain metastase from melanoma, lung, breast, kidney primary cancers.

In specific embodiments, said subject is selected from human patient with multiple brain metastases. In other specific embodiments, said subject is selected from human patient with multiple brain metastases ineligible for local treatment by surgery or stereotactic radiation.

The method of the present disclosure for treating cancer comprises at least two steps of administering an efficient amount of said nanoparticles to the tumor of the subject.

The nanoparticles can be administered to the subject using different possible routes such as local (intra-tumoral (IT), intra-arterial (IA)), subcutaneous, intravenous (IV), intradermic, airways (inhalation), intra-peritoneal, intramuscular, intra-thecal, intraocular or oral route.

In specific embodiments, the nanoparticles are administered intravenously. Indeed, the nanoparticles as disclosed herein are advantageously targeted to the human tumors, by passive targeting, for example by enhanced permeability and retention effect.

Further injections or administrations of nanoparticles can be performed, when appropriate. Typically, when using fractionated radiation therapy, the nanoparticles may be further injected once every week during the radiation therapy. For example, in a specific embodiment, the method as disclosed herein further comprises a step of injecting a third therapeutically effective amount of the same or different high-Z element containing nanoparticles within 5-10 days after the second injecting step, for example 7 days after the second injecting step.

In specific embodiments, said nanoparticles are gadolinium-chelated polysiloxane based nanoparticles and a therapeutically effective amount administered intravenously at each injecting step is comprised between 50 mg/kg and 150 mg/kg, typically, between 80 and 120 mg/kg, for example 100 mg/kg.

Advantageously, the same nanoparticles may be used as theranostics for detection of the tumors by MRI, prior to its use as radiosensitizing agent. Accordingly the method further comprises a step of imaging the tumor by magnetic resonance imaging (MRI) after the first injecting step of said nanoparticles, wherein said nanoparticles is used as a T1 contrast agent for said MRI. The results in the examples provide evidence of an excellent signal enhancement in human patients for detecting tumors by MRI, for example brain metastases, after injection of said high-Z containing nanoparticles.

The Step of Irradiating the Tumor

The methods of use of the nanoparticles include a step of irradiating the tumor of subject in need thereof, for radiation therapy of the corresponding cancer, wherein said nanoparticles advantageously enhance the dose efficacy of the radiation therapy.

As used herein, radiation therapy or radiotherapy is the medical use of irradiation—i.e. ionizing radiation—as part of cancer treatment to control malignant cells. It is used as palliative treatment or as therapeutic treatment. Radiotherapy is accepted as an important standard therapy for treating various types of cancers.

As used herein, the term “radiotherapy” is used for the treatment of diseases of oncological nature with irradiation corresponding to ionizing radiation. Ionizing radiation deposits energy that injures or destroys cells in the area being treated (the target tissue) by damaging their genetic material, making it impossible for these cells to continue to grow.

In specific embodiment, the method of the disclosure comprises exposing the tumor to be treated to an efficient dose of ionizing radiations, wherein said ionizing radiations are photons, e.g. X-rays. Depending on the amount of energy they possess, the rays can be used to destroy cancer cells on the surface of or deeper in the body. The higher the energy of the X-ray beam, the deeper the X-rays can go into the target tissue. Linear accelerators and betatrons produce X-rays of increasingly greater energy. The use of machines to focus radiation (such as X-rays) on a cancer site is called external beam radiotherapy.

In an alternative embodiment of the method of treatment according to the disclosure, gamma rays are used. Gamma rays are produced spontaneously as certain elements (such as radium, uranium, and cobalt 60) release radiation as they decompose, or decay.

Ionizing radiations are typically of 2 keV to 25000 keV, in particular of 2 keV to 6000 keV (i.e. 6 MeV) or of 2 keV to 1500 keV (such as cobalt 60 source).

A person of ordinary skill in the radiotherapy art knows how to determine an appropriate dosing and application schedule, depending on the nature of the disease and the constitution of the patient. In particular, the person knows how to assess dose-limiting toxicity (DLT) and how to determine the maximum tolerated dose (MTD) accordingly.

The amount of radiation used in radiation therapy is measured in gray (Gy), and varies depending on the type and stage of cancer being treated. For curative cases, the typical total dose for a solid tumor ranges from 20 to 120 Gy. Many other factors are considered by radiation oncologists when selecting a dose, including whether the patient is receiving chemotherapy, patient co-morbidities, whether radiation therapy is being administered before or after surgery, and the degree of success of surgery.

The total dose is typically fractionated (spread out over time). Amount and schedules (planning and delivery of ionizing radiations, fraction dose, fraction delivery schema, total dose alone or in combination with other anti-cancer agents etc) is defined for any disease/anatomical site/disease stage patient setting/age and constitutes the standard of care for any specific situation.

A typical conventional fractionation schedule for adults for the methods of the present disclosure may be 2.5 to 3.5 Gy per day, five days a week, for example for 2 to 8 consecutive weeks. In specific embodiments, said radiotherapy consists of exposing the subject to a total dose of ionizing radiations between 25 and 35 Gy, for example 30 Gy.

In other specific embodiments, the subject is exposed to a dose of ionizing radiations per fraction of about 2 to 8 Gy, and the total dose is administered preferably in a maximum of 10 fractions.

In specific embodiment where the subject is suffering from brain cancer, for example multiple brain metastases, the radiation therapy applied at step (iii) of the herein disclosed methods is a whole brain radiation therapy (WBRT). The most common dose/fractionation schedule used in WBRT is 30 Gy delivered in 10 fractions over the course of 2 weeks as used for example in the Example 2.

Combination Therapy with the Method of the Present Disclosure

The nanoparticles for use as disclosed herein may be administered as the sole active ingredient or in conjunction with, e.g. as an adjuvant to or in combination to, other drugs e.g. cytotoxic, anti-proliferative, or other anti-tumor agents, e.g. for the treatment or prevention of cancer disorders, as mentioned above.

Suitable cytotoxic, anti-proliferative or anti-tumor agents may include without limitation cisplatin, doxorubicin, taxol, etoposide, irinotecan, topotecan, paclitaxel, docetaxel, epothilones, tamoxifen, 5-fluorouracil, methotrexate, temozolomide, cyclophosphamide, tipifarnib, gefitinib, erlotinib, imatinib, gemcitabine, uracil mustard, chlormethine, ifosfamide, melphalan, chlorambucil, pipobroman, triethylenemelamine, busulfan, carmustine, lomustine, streptozocin, dacarbazine, floxuridine, cytarabine, 6-mercaptopurine, 6-thioguanine, fludarabine phosphate, oxaliplatin, folinic acid, pentostatin, vinblastine, vincristine, vindesine, bleomycin, dactinomycin, daunorubicin, epirubicin, idarubicin, mithramycin, deoxycoformycin, mitomycin-C, L-asparaginase, teniposide.

In some embodiments, the additional therapeutic agent is administered simultaneously with a composition provided herein. In some embodiments, the additional therapeutic agent is administered after administration of the composition provided herein. In some embodiments, the additional therapeutic agent is administered prior to administration of the composition herein. In some embodiments, the composition provided herein is administered during a surgical procedure. In some embodiments, the composition provided herein is administered in combination with an additional therapeutic agent during a surgical procedure.

The additional therapeutic agents provided herein can be effective over a wide dosage range and are generally administered in an effective amount. It will be understood, however, that the amount of the therapeutic agent actually administered will usually be determined by a physician, according to the relevant circumstances, including the condition to be treated, the chosen route of administration, the actual compound administered, the age, weight, and response of the individual subject, the severity of the subject's symptoms, and the like.

Other aspects and advantages of the method of the disclosure will become apparent in the following examples, which are given for purposes of illustration only.

### EXAMPLES

### Example 1: First-In-Human Trial of Gd-Based Theranostic Nanoparticles: Uptake and Biodistribution in Patients with 4 Types of Brain Metastasis

1.1 Materials and Methods

Study Design

This study is part of a prospective dose escalation phase I-b clinical trial to evaluate the tolerance of the intravenous administration of radiosensitizing AGuIX nanoparticles in combination with whole brain radiotherapy for the treatment of brain metastases. The Nano-Rad trial (Radiosensitization of Multiple Brain Metastases Using AGuIX Gadolinium Based Nanoparticles) was registered as NCT02820454. Here, we report the findings of the MRI protocol applied to the 15 recruited patients. The objectives assigned to this MRI ancillary study were to i) assess the distribution of AGuIX nanoparticles in brain metastases and surrounding healthy tissues and ii) to measure the T₁-weighted contrast enhancement and nanoparticle concentration in brain metastases and surrounding healthy tissues after intravenous administration of AGuIX nanoparticles (Verry C, et al. BMJ Open. 9:e023591 (2019)).

Patient Selection

Patients with multiple brain metastases ineligible for local treatment by surgery or stereotactic radiation were recruited. Inclusion criteria included: i) minimum age of 18 years, ii) secondary brain metastases from a histologically confirmed solid tumor, iii) no prior brain irradiation, iv) no renal insufficiency (glomerular filtration rate >60 mL/min/1.73 m²), v) normal liver function (bilirubin <30 μmol/L; Alkaline phosphatase <400 UI/L; Aspartate aminotransferase (AST) <75 UI/L; Alanine aminotransferase (ALT) <175 UI/L).

Trial Design

The main steps of the trial protocol were as follows. At D0, patients underwent a first imaging session (see MRI protocol in next paragraph) including the intravenous bolus injection of Dotarem (gadoterate meglumine) at a dose of 0.2 mL/kg (0.1 mmol/kg) body weight. 1 to 21 days after the first imaging session (depending on patient availability and radiation therapy planning), the patients were administered intravenously with solution of AGuIX nanoparticles at doses of 15, 30, 50, 75 or 100 mg/kg body weight. The date of AGuIX nanoparticles administration is referred as D1.

Synthesis of AGuIX Nanoparticles

AGuIX nanoparticles have been obtained by a six step synthesis. The first step is the formation of a gadolinium oxide core by addition of soda on gadolinium trichloride in diethylene glycol. Second step is growth of a polysiloxane shell by adding TEOS and APTES. After maturation, DOTAGA anhydride is added for reaction with free amino functions present at the surface of the inorganic matrix. After transfer to water, dissolution of the gadolinium oxide core is observed and gadolinium is chelated by DOTAGA at the surface of the matrix. Then, fragmentation of the polysiloxane matrix in ultrasmall AGuIX nanoparticles is observed. Last step is freeze drying of the nanoparticles.

The theranostic agent is composed of a polysiloxane network surrounded by gadolinium cyclic ligands, derivatives of DOTA (1,4,7,10-tetraazacyclododecane acid-1,4,7,10-tetraacetic acid), covalently grafted to the polysiloxane matrix. Its hydrodynamic diameter is 4±2 nm, its mass is about 10 kDa and is described by the average chemical formula (GdSi₄₋₇C₂₄₋₃₀N₅₋₈O₁₅₋₂₅H₄₀₋₆₀, 5-10 H₂O)ₓ. On average each nanoparticle presents on its surface 10 DOTA ligands which chelate core gadolinium ions. The longitudinal relaxivity r₁ at 3 Tesla is equal to 8.9 mM⁻¹·s⁻¹ per Gd³⁺ ion, resulting in a total r₁ of 89 mM⁻¹·s⁻¹ per AGuIX nanoparticle. The same MRI session, without injection of gadoterate meglumine, was performed 2 hours post administration of the nanoparticles. The patients then underwent a whole brain radiation therapy (30 Gy delivered in 10 sessions of 3 Gy). 7 days (D8) and 4 weeks (D28) after the AGuIX nanoparticles were administered, a similar MRI session was performed for each patient.

MRI Protocol

The MRI acquisitions were performed on a 3 Tesla Philips scanner. 32-channel Philips head coil were used. Patients underwent identical imaging protocol including the following MRI sequences: i) 3D T₁-weighted gradient echo sequence, ii) 3D FLASH sequence with multiple flip angles, iii) susceptibility-weighted imaging (SWI) sequence, iv) Fluid Attenuated Inversion Recovery (FLAIR) sequence, v) Diffusion-weighted imaging (DWI) sequence. Some of these imaging sequences are recommended when following the RECIST (Response Evaluation Criteria in Solid Tumors) and RANO (Response Assessment in Neuro-Oncology) criteria for assessing brain metastases response after radiotherapy (24, 25). The 3D T₁-weighted imaging sequence provides high-resolution contrast-enhanced images of healthy tissue and brain metastases following MRI contrast agent administration. The 3D FLASH sequence is repeated several times with a different flip angle for computing T₁ relaxation times and contrast agent concentration. The SWI sequence is used for detecting the presence of hemorrhages. The FLAIR sequence is applied for monitoring the presence of inflammation or edema. Finally, the DWI sequence can be applied for detecting abnormal water diffusion in tissue or brain metastases. The total acquisition time ranged between 30 minutes and 40 minutes depending on patient-adjusted imaging parameters. The key features and the main acquisition parameters of these imaging sequences are detailed in Supplementary Materials section.

Image Processing and Quantification Pipeline

MRI analyses were performed using an in-house computer program called MP³ (https://github.com/nifm-gin/MP3) developed by the GIN Laboratory (Grenoble, France) and running under Matlab® software. Image analyses include counting and measurements of metastases, quantification of contrast enhancement, relaxation times and concentration of nanoparticles. Following RECIST and RANO criteria, solely metastases with longest diameter above 1 cm were considered as measureable and were retained in subsequent analyses. The MRI enhancement, expressed in percentage, was defined as the ratio of the MRI signal amplitude post contrast agent administration over the MRI signal amplitude pre contrast agent administration; the MRI signal amplitude being measured in the 3D T₁-weighted image dataset. The T₁ relaxation times were derived from the 3D FLASH images obtained at four different flip angles. The concentration of nanoparticles in brain metastases was derived from the variations of T₁ relaxation times pre and post contrast agent administration and from the known relaxivity of the nanoparticles.

A 3D image rendering was performed using the BrainVISA/Anatomist software (http://brainvisa.info) developed at NeuroSpin (CEA, Saclay, France). To better visualize the location of the different metastases, the Morphologist pipeline of BrainVISA was used to generate the meshes of both the brain and the head of each patient.

Statistical Analysis

All analyses were performed using GraphPad Prism (GraphPad Software Inc.). Unless specified, significance was fixed at a 5% probability level. Unless specified, all of the data are presented as mean value ±SD.

1.2 Results

Administered AGuIX Gd-Based Nanoparticles Induce MRI Contrast Enhancement in all Four Types of Brain Metastases

The patient recruitment resulted into the inclusion of four types of brain metastases, namely NSCLC (non-small-cell lung carcinoma) N=6, breast N=2, melanoma N=6 and colon cancer N=1. All the patients were successfully injected with the theranostic nanoparticles AGuIX (as described in Materials and Methods) at each escalation step of administered dose (N=3 for 15, 30, 50, 75 and 100 mg/kg body weight).

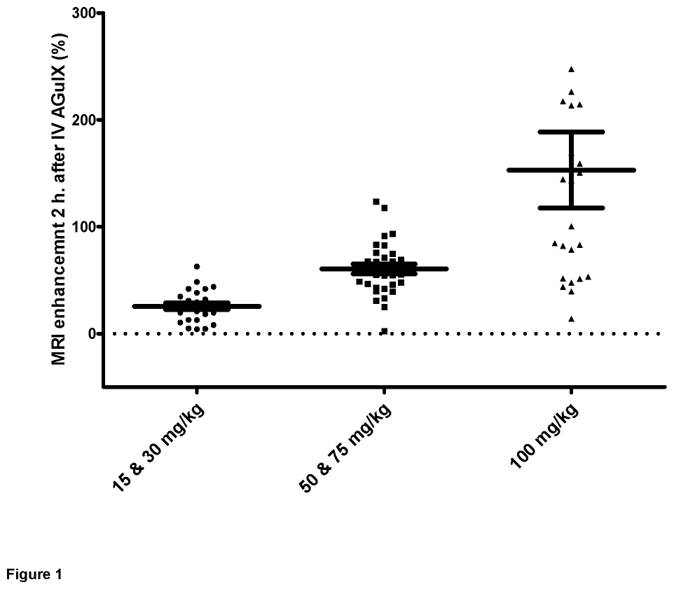

At D1, two hours after AGuIX injection, MRI signal enhancements were observed for all types of brain metastases, all patients and all doses administered. Within the region of interest drawn around each metastasis, MRI signal enhancements were found to increase with the administered dose of AGuIX nanoparticles (FIG. 1). Signal enhancements, averaged over all measurable metastases (longest diameter greater than 1 cm), were equal to 26.3±15.2%, 24.8±16.3%, 56.7±23.8%, 64.4±26.7% and 120.5±68% for AGuIX doses of 15, 30, 50, 75 and 100 mg/kg body weight respectively. The MRI enhancement was found to linearly correlate with the injected dose (slope 1.08, R²=0.90) (data not shown).

Gd-Based Nanoparticles Demonstrate MRI Enhancement of Brain Metastases Equivalent to that of a Clinically-Used Contrast Agent

For each patient, the MRI enhancement was also measured at D0 15 min after injection of a clinically-approved Gd-based contrast agent (Dotarem®, Guerbet, Villepinte, France). Averaged over all measurable metastases with longest diameter larger than 1 cm, the MRI enhancement was equal to 182.9±116.2%. This MRI enhancement, observed 15 min after injection, is in the same order of magnitude than the one observed 2 h after the administration of the highest dose of AGuIX nanoparticles.

The detection sensitivity of AGuIX nanoparticles, defined as their ability of enhancing MRI signal in measurable brain metastases was assessed for all administered doses and compared to the sensitivity of the clinically-used contrast agent Dotarem®. Expressed as a percentage of Dotarem sensitivity, the AGuIX nanoparticles sensitivity was equal to 12.1, 19.5, 34.2, 31.8 and 61.6% or injected doses of 15, 30, 50, 75 and 100 mg/kg body weight respectively.

Concentration of AGuIX Nanoparticles can be Quantified in Brain Metastases

The multi flip-angle 3D FLASH acquisitions were successfully used to compute pixel-wise maps of T₁ values (data not shown) and to enable quantification of the longitudinal relaxation time over regions of interest. The decrease of T₁ relaxation times in brain metastases, induced by the uptake of AGuIX nanoparticles, is clearly shown in these T₁ maps. As expected, the decreases of T₁ values are co-localized with the contrast-enhanced brain metastases.

The concentrations of AGuIX nanoparticles in contrast-enhanced metastases were computed based on changes in T₁ values following their administration. The measurements of AGuIX concentration were performed in metastases with longest diameter larger than 1 cm for the patients administered with a dose of 100 mg/kg body weight. The mean AGuIX concentration in the brain metastases was measured to be 57.5±14.3, 20.3±6.8, 29.5±12.5 mg/L in patient #13, #14 and #15 respectively.

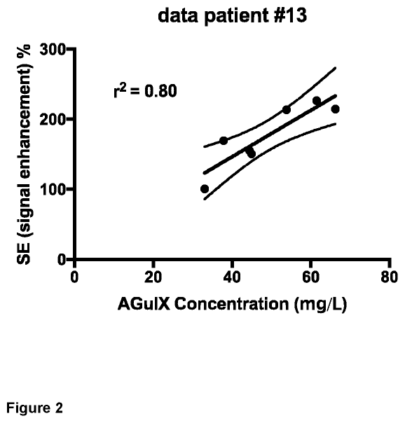

The correlation between MRI enhancement and nanoparticles concentration was assessed for patients with the highest (100 mg/kg) administered dose. The correlation is exemplified in FIG. 2 with MRI data from patient #13 with NSCLC metastases. A strong positive correlation between the two MRI parameters was observed with a relationship close to linearity in the range of measured values.

For each patient, the MRI enhancement and T₁ values were assessed in brain regions of interest free of visible metastases (three representative regions of interest per patient, with a similar size for all patients). No significant MRI enhancement and no T₁ variations were observed in any of these healthy brain regions.

MRI Enhancements are Observed One Week after Nanoparticle Administration

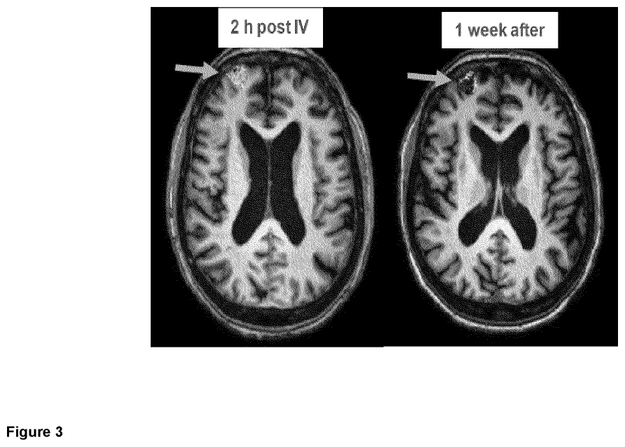

For patients administered with the largest dose (100 mg/kg body weight), persistence of MRI enhancement was noticed in measurable metastases (longest diameter greater than 1 cm) at D8, i.e. up to one week after administration of AGuIX nanoparticles as shown in FIG. 3. The mean MRI enhancements in metastases were measured equal to 32.4±10.8%, 14±5.8% and 26.3±9.7% for patient #13, #14 and #15 respectively. As a point of comparison, the mean MRI enhancements at D1 were equal to 175.8±45.2%, 58.3±18.4% and 154.1±61.9% for patients #13, #14 and #15 respectively. Due to low T₁ variations, the concentration of AGuIX nanoparticles could not be computed. Based on the observed correlation between MRI enhancement and nanoparticles concentration, an upper limit of 10 □M can be estimated for the AGuIX concentration at D8 in brain metastases. No noticeable MRI enhancement was observed in any patient at D28, 4 weeks after the administration of AGuIX nanoparticles.

### DISCUSSION

The occurrence of brain metastases is a common event in the history of cancer and negatively affects the life expectancy of patients. For patients with multiple brain metastases, whole brain radiation therapy (WBRT) remains the standard of care. However, the median overall survival is less than six months and new approaches need to be developed to improve the treatment efficacy for these patients. The use of radiosensitizing agents is thus of great interest. The in vivo theranostic properties (radiosensitization and diagnosis by multimodal imaging) of AGuIX nanoparticles were previously demonstrated in preclinical studies performed on eight tumor models in rodents (F. Lux, et al. Br J Radiol. 18:20180365 (2018)), and particularly in brain tumors (G. Le Duc, et al. ACS Nano. 5, 9566-9574 (2011), C. Verry C, et al. Nanomedicine 11, 2405-2417 (2016)). The clinical evaluation of the diagnostic value of the AGuIX nanoparticles for brain metastases was one of the secondary objectives of the clinical trial Nano-Rad. The target dose for the radiotherapeutic application of the AGuIX nanoparticles in patients is 100 mg/kg and for this reason the conclusions and perspectives of this study focus essentially on this dose.

The largest dose of AGuIX nanoparticles (100 mg/kg body weight or 100 μmol/kg body weight Gd³⁺) administered to the patients corresponds to the amount of chelated gadolinium ions Gd³⁺ injected in one dose of clinically-used MRI contrast agent such as Dotarem® (100 □mol/kg body weight Gd³⁺). It is therefore appropriate to compare the MRI enhancements observed in metastases with the largest AGuIX dose to a dose of Gd-based contrast agent used in clinical routine.

In this study, there was a 2-hours delay between the nanoparticle administration and the MRI acquisitions for monitoring the patient's response to the injection. With a mean nanoparticle plasma half-life of about 1 hour, this delay results in an 86% decrease of the nanoparticles concentration in the plasma. In contrast, there was only a 15-minutes delay between the Dotarem® injection and the MRI acquisition. Despite this significant clearance of nanoparticles and the decrease of concentration in the patient's bloodstream, the MRI enhancement at the highest nanoparticle dose is close to that observed with the clinical contrast agent.

This remarkable diagnostic performance of AGuIX nanoparticles to enhance the MRI signal in brain metastases may be attributed to two independent factors. The first factor is related to the intrinsic magnetic properties of nanoparticles. Their larger diameter and molecular weight, as compared to clinical Gd-based contrast agent, result in a higher longitudinal relaxation coefficient r₁ and thus an increased ability to modify the intensity of the MRI signal. Concretely, the r₁ values for AGuIX nanoparticles and Dotarem® are respectively equal to 8.9 and 3.5 mM⁻¹·s⁻¹ per Gd³⁺ ion at a magnetic field of 3 Tesla (B. R. Smith, S. S. Gambhir. Chem Rev. 117, 901-986 (2017)).

The second factor may be related to the ability of AGuIX nanoparticles to passively accumulate in brain metastases. This passive targeting phenomenon takes advantage of the so-called Enhanced Permeability and Retention (EPR) effect which postulates that the accumulation of nano-objects in tumors is due to both defective and leaky tumor vessels and to the absence of effective lymphatic drainage (A. Bianchi, et al. MAGMA. 27, 303-316 (2014)). The passive targeting of tumors by AGuIX nanoparticles has been consistently observed in previous investigations of animal models of cancer. In a mouse model of multiple brain melanoma metastases, internalization of AGuIX nanoparticles in tumor cells was reported and the presence of nanoparticles in brain metastases was still observed 24 hours after intravenous injection to the animals (Kotb, A. et al. Theranostics 6(3):418-427 (2016)). At the highest 100 mg/kg dose, all metastases with a diameter larger than 1 cm were contrast-enhanced up to 7 days after the nanoparticles were administered.

The persistence of MRI signal enhancement in metastases one week after administration highlights this accumulation and delayed clearance of nanoparticles from the metastasis. To the best of the inventor's knowledge, there is no report in the literature of such late MRI enhancement in metastases after administration of clinically used Gd-based contrast agents.

A dose escalation was included in the design of this first-in-man clinical trial and the patients were thus administered with five increasing dose levels of AGuIX nanoparticles. From the linear correlation observed between the signal enhancement in metastases and the administered nanoparticles concentration, it can be concluded that the dose of nanoparticles—in the range of investigated doses—is not a limiting factor for the passive targeting of metastases. Importantly, despite the limited number of patients participating in this first clinical study, these initial results show that nanoparticles uptake and signal enhancement are present in the four types of investigated metastases (NSCLC, melanoma, breast and colon), regardless of the injected dose of nanoparticles.

Considering the radiosensitizing properties of AGuIX nanoparticles, it is key to evaluate and possibly quantify the local concentration of nanoparticles accumulated in metastases. To that end, the MRI protocol included a T₁ mapping imaging sequence from which the nanoparticles concentration was derived. The concentration values obtained in this clinical study can be put in perspective with those obtained in pre-clinical studies in animal models of tumor. The computed concentration of AGuIX nanoparticles in the NSCLC and breast cancer metastases of the three patients injected with the highest dose varied between 8 and 63 mg/L, corresponding to a concentration range of Gd³⁺ ions between 8 and 63 □M in brain metastases. In the three aforementioned patients, the % ID/g is ranging between 8 and 63%. The same order of magnitude was found for the % ID/g in the two above-mentioned MRI pre-clinical studies, with 28% and 45% ID/g respectively. Interestingly, these concentrations are obtained with a delay post-injection (several hours) that is compatible with the setup of a radiotherapy session.

In this study, we evaluated as well the relationship between the nanoparticles concentration and the MRI signal enhancement SE obtained using a robust T₁-weighted 3D MRI sequence. In the range of measurable nanoparticles concentration in metastases, a linear relationship between the MRI enhancement and the nanoparticles concentration is observed with the acquisition protocol used in this study. Hence, with the specific protocol used in this study, the MRI enhancement can be used as a robust and simple index for assessing the concentration of AGuIX nanoparticles.

While metastasis targeting is beneficial for both diagnosis and radiosensitization purposes, it is desirable to maintain nanoparticles at low concentration in healthy surrounding tissues. In this respect, no MRI enhancement could be observed in the metastasis-free brain tissues two hours after the highest dose of AGuIX nanoparticles was administered. This lack of enhancement is consistent with the rapid clearance of nanoparticles measured in patient's plasma and is a positive indication of the innocuousness of the nanoparticles for the healthy brain.

In summary, the preliminary results of the clinical trial reported here demonstrate that an intravenous injection of Gd-based nanoparticles is effective for enhancing different types of brain metastases in patients. These first clinical findings—pharmacokinetic, passive targeting, concentration in metastases—are in line with the observations obtained in previous pre-clinical studies in animal models of brain tumor and bodes well for a successful translation of this theranostic agent from the preclinical to the clinical level.

In addition to this, the preliminary results of the Nano-Rad phase 1 clinical trial demonstrate a good tolerance of intravenous injection of AGuIX nanoparticle up to the 100 mg/kg dose selected for this study.

Lastly, the persistence of the nanoparticles in the tumor at D8 support a protocol including a first injection within a period between 2 and 7 days prior to the first irradiation, in order to optimize the concentration and distribution of the tumors within the tumor while minimizing the presence of the nanoparticles in the surrounding healthy tissues.

All these results and observations provide strong and credible support for a phase 2 clinical trial (NANORAD2, NCT03818386) as disclosed in the Example 2 hereafter.

### Example 2: Radiotherapy of Multiple Brain Metastases Using AGuIX Gadolinium-Chelated Polysiloxane Based Nanoparticles: A Prospective Randomized Phase II Clinical Trial

2.1 Research Hypotheses and Expected Results

The preliminary results of the phase lb trial Nanorad have confirmed the interest of the combination of AGuIX nanoparticles with radiation therapy for the treatment of cancer patients, and especially patients with brain metastases.

The purpose of this randomized phase II study will be to evaluate the efficacy of the combination of AGuIX with WBRT to demonstrate an increase in intracranial response rate compared to WBRT alone. Moreover, an increase in intracranial progression-free survival is expected, as well as an improvement of quality of life for the patients. In addition, as gadolinium is a positive contrast agent T₁ in MRI, an MRI study will be performed to evaluate the distribution of the product in brain metastases and the surrounding healthy tissues.

The target population chosen is patients with brain metastases eligible for treatment by whole brain radiation therapy, regardless of their primary cancer.

The target population was selected on:
The relevance of the intravenous administration of a radiosensitizer on the multifocal nature of brain metastases;
The differential of tissue enhancement between tumor lesions and healthy brain in preclinical in vivo studies, as well as for patients included in the phase I trial Nanorad (EudraCT 2015-004259-30; NCT02820454);
The absence of a therapeutic alternative for those patients whose life expectancy is about 4.5 months with standard radiotherapy treatment.

Other tumor sites could also benefit from an increase of the dose efficacy related to the radiosensitizing effect of the nanoparticles.

2.2 Design/Methodology of the Study

This is a Prospective Randomized Open Blinded End-point phase II clinical trial to evaluate the efficacy of AGuIX in combination with whole brain radiotherapy in comparison to whole brain radiotherapy alone for the treatment of brain metastases (Blood Press. 1992 August; 1(2):113-9. Prospective randomized open blinded end-point (PROBE) study. A novel design for intervention trials. Prospective Randomized Open Blinded End-Point. Hansson L) (Hansson et al., 1992).

Patients will be allocated to one of the two groups of treatment following a minimization procedure that will be performed centrally, in order to balance potential prognostic factors between the treatment groups. The minimization algorithm will take into account the following factors: center, age (as a continuous variable), histology of primary cancer (lung versus breast versus melanoma versus other), DS-GPA score (≤1versus>1), history of intracranial local treatment (yes versus no), immunotherapy treatment (yes versus no), treatment with corticoids at inclusion (yes versus no), intention to performed hippocampal protection (yes versus no).

The study will be adaptive: an interim analysis is planned after enrolment of 20 patients in each arm of treatment (WBRT and AGuIX+WBRT) in order to drop an a priori identified subgroup should there be no difference between groups. The two identified groups for the interim for the selection are (i) patients with melanoma brain metastases and (ii) patients with brain metastases from all other primary cancers (lung, breast, kidney . . . ).

In each of the two subgroups, a contingency table will describe the response rates and any group with no or worse response (i.e. Best objective intracranial response rate equal or worse than the reference group) may be dropped.

2.3 Eligibility Criteria/Subject cHaractEristics

Inclusion Criteria
Patients with brain metastases, from a histologically confirmed solid tumor, eligible for WBRT
At least 18 years old
Signed informed consent
ECOG (Eastern Cooperative Oncology Group) performance status 0-2
Extracranial disease:
Complete or partial response or stability under systemic treatment
No extracranial disease
Or first line of treatment
Life expectancy greater than 6 weeks
Effective contraceptive method for all patient of childbearing potential
Affiliated to a social security regimen or beneficiary of the same

Non-inclusion criteria
Leptomeningeal metastasis
Evidence of metastasis with recent large hemorrhage
Progressive and threatening extracranial disease under systemic treatment
Previous cranial irradiation (except stereotactic irradiation)
Known contra-indication, sensitivity or allergy to gadolinium
Known contra-indication for Magnetic Resonance Imaging
Renal insufficiency (glomerular filtration rate 50 mL/min/1.73 m²)
Inclusion in another clinical trial protocol
Pregnancy or breastfeeding
Subject in exclusion period of another study
Subject under administrative or judicial control

2.4 Allowed Treatment(s)/Procedure(s)

Maintaining or stopping of systemic cancer treatments prior to cerebral irradiation is left to the discretion of each investigator and must comply with current recommendations.

The following treatments are contra-indicated during the radiotherapy: GEMZAR®, AVASTIN®, VEMURAFENIB® as well as Tyrosine Kinase Inhibitors. They must be stopped before radiation according to current recommendations.

Apart from those restrictions, the usual treatments of the patients are allowed and will be listed in the CRF of each patient.

As a precaution, radiotherapy should not be performed within less than 2 hours after AGuIX administration. The plasmatic half-life of the particles is 1.08 hour in humans (0.75 to 2 hours). Too early radiotherapy may increase the effect of radiotherapy in healthy brain tissues even though in animal models the amounts of nanoparticles in healthy brain tissues remain low (Verry, C., et al. (2016). MRI-guided clinical 6-MV radiosensitization of glioma using a unique gadolinium-based nanoparticles injection. Nanomedicine (Lond).

2.5 Treatment(s) Used in the Study

Name of drug/treatment and trade name: AGuIX

Chemical name (DCI): Gadolinium-chelated polysiloxane based nanoparticles

Pharmaceutical form: sterile lyophilized off-white powder containing 300 mg AGuIX as active ingredient (300 mg of AGuIX/vial). Each vial contains 0.66 mg of CaCl₂), as an inactive ingredient. The drug product is supplied in a single-use 10 mL clear glass vial with a bromobutyl rubber stopper.

Preparation procedure: reconstitution of the solution with 3 mL of water for injection to obtain a solution of AGuIX at 100 mg/mL. The pH of the solution is then at 7.2±0.2.

One hour after reconstitution with water for injection, the reconstituted solutions will be taken into a syringe, before being injected using a syringe pump.

Administration minimum 1 hour after reconstitution and within 24 hours maximum.

AGuIX solution will be administered in a half-day after reconstitution, however, the nanoparticles must be stored at [+2° C.; +8° C.] and administered within a maximum of 24 hours after reconstitution.

Intravenous administration by slow infusion (2 mL/min) with a syringe pump

Dose per administration: 100 mg/kg, 1 mL/kg

2.6 Treatment and Associated Procedures

Radiotherapy: Whole Brain Radiation Therapy:
Dosimetric scanner and individual contention mask
Irradiation techniques allowed:
   conformational, isometric, 2 lateral beams of 6 MV photons
   or IMRT (tomotherapy or VMAT)
Dose
   Prescription dose: 30 Gy
   Dose per fraction: 3 Gy
   Number of fractions: 10
   Fraction per day: 1
   Total treatment duration should not exceed 3 weeks

Target Volume:
Clinical Target Volume (CTV)=encephalon+brainstem+cerebellum
Planning Target Volume (PTV)=CTV+3 mm

Organs at Risk:
Eye, lens, retina, hippocampus, internal ear and cochlea

Dose Constraints:
PTV: V95%>95, D max<107%

Hippocampal Sparing:

Hippocampal sparing is recommended when possible but not mandatory. In case of intensity modulation irradiation with hippocampal sparing, it is necessary to make fusion between the dosimetric scanner and the reference MRI. Bilateral hippocampal contours were manually generated on the fused MRI-CT image set and expanded by 5 mm to generate the hippocampal avoidance regions. The planning target volume (PTV) was defined as the clinical target volume excluding the hippocampal avoidance regions. IMRT was delivered to a dose of 30 Gy in 10 fractions to cover the PTV while avoiding the hippocampus. Dose to 100% of the hippocampus could not exceed 9 Gy, and maximal hippocampal dose could not exceed 16 Gy.

2.7 In-Treatment Visits
Experimental treatment arm: AGuIX+WBRT

Day 0 (Between 2 to 7 Days Before WBRT Start):
Patient hospitalized at the Ambulatory Unit
Clinical exam with determination of ECOG OMS Performance status, pain, weight and constants (Heart frequency, Blood pressure, Temperature)
Neurological exam
Placement of a peripheral venous catheter for AGuIX® injection
Biological exams: Complete blood counts, serum electrolytes, renal function, liver function (see table § 7.3 and § 8.3 for details)
First administration of AGuIX (H0) between 2 to 7 days before WBRT start
Brain MRI performed within one hour after injection*
Safety assessment

Week 1 of Radiotherapy Treatment:

Session 1:
Patient hospitalized at the Ambulatory Unit
Clinical exam with determination of ECOG OMS Performance status, pain, weight and constants (Heart frequency, Blood pressure, Temperature)
Neurological exam
Placement of a peripheral venous catheter for AGuIX® injection
Biological exams: Complete blood counts, serum electrolytes, renal function, liver function (see table § 7.3 and § 8.3 for details). The physician must verify that the patient has no change in its renal function prior performing AGuIX® injection.
Second administration of AGuIX (H0)
Radiotherapy session no 1, performed between 3 to 5 hours after second administration of AGuIX injection
(as a precaution, radiotherapy should not be performed within less than 2 hours after AGuIX administration)
Safety assessment

Session 2/Session 3/Session 4/Session 5:

Ambulatory radiotherapy sessions no 2, no 3, no 4 and no 5

Week 2 of Radiotherapy Treatment:

Session 6 (before radiotherapy session no 6):
Patient hospitalized at the Ambulatory Unit
Clinical exam with determination of ECOG OMS Performance status, pain, weight and constants (Heart frequency, Blood pressure, Temperature)
Neurological exam
Placement of a peripheral venous catheter for AGuIX® injection
Biological exams: Complete blood counts, serum electrolytes, renal function, liver function (see table § 7.3 and § 8.3 for details). The physician must verify that the patient has no change in its renal function prior performing AGuIX injection.
Third administration of AGuIX (H0)
Radiotherapy session no 6, performed between 3 to 5 hours after AGuIX injection.
(As a precaution, radiotherapy should not be performed within less than 2 hours after AGuIX administration)
Safety assessment

Session 7/Session 8/Session 9/Session 10:
Ambulatory radiotherapy sessions no 7, no 8, no 9 and no 10
   Control treatment arm: WBRT

Week 1 of Radiotherapy Treatment

Session 1:
Clinical exam with determination of ECOG OMS Performance status, pain, weight and constants (Heart frequency, Blood pressure, Temperature)
Neurological exam
Biological exams: Complete blood counts, serum electrolytes, renal function, liver function (see table § 7.3 and § 8.3 for details).
Radiotherapy session no 1
Safety assessment

Session 2/Session 3/Session 4/Session 5:

Ambulatory radiotherapy sessions no 2, no 3, no 4 and no 5

Week 2 of Radiotherapy Treatment:

Session 6 (Before Radiotherapy Session no 6):
Clinical exam with determination of ECOG OMS Performance status, pain, weight and constants (Heart frequency, Blood pressure, Temperature);
Neurological exam;
Biological exams: Complete blood counts, serum electrolytes, renal function, liver function (see table § 7.3 and § 8.3 for details);
Radiotherapy session no 6;
Safety assessment.

Session 7/Session 8/Session 9/Session 10:
Ambulatory radiotherapy sessions no 7, no 8, no 9 and no 10

Follow-Up Visits

The follow-up visits schedule will be the same whatever the treatment arm (i.e. experimental treatment arm AGuIX+WBRT and control treatment arm WBRT).

The follow-up visits will take place at 6 weeks and then at 3, 6, 9 and 12 months after radiotherapy commencement (+/− one week).

End of Study Visit

The end of study will take place 12 months after radiotherapy commencement for the two treatment arms (i.e. experimental treatment arm AGuIX+WBRT and control treatment arm WBRT).

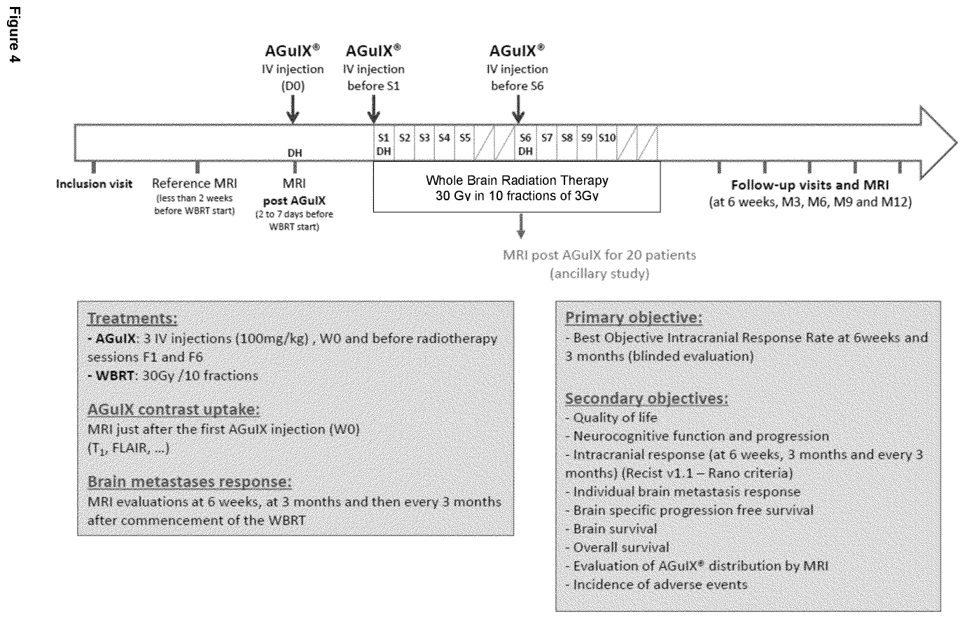

The study protocol and objectives are also summarized in FIG. 4.

Results

The clinical trial using the treatment protocol described in this example 2 was initiated. To date, 10 patients receiving several administrations of AGuIX nanoparticles have been enrolled with no major adverse effects reported.

### REFERENCES

1. Schaue D, McBride W H. Opportunities and challenges of radiotherapy for treating cancer. Nat Rev Clin Oncol 2015, 12(9): 527-540.
2. Beasley M, Driver D, Dobbs H J. Complications of radiotherapy: improving the therapeutic index. Cancer Imaging 2005, 5: 78-84.
3. Hainfeld J F, O'Connor M J, Dilmanian F A, Slatkin D N, Adams D J, Smilowitz H M. Micro-CT enables microlocalisation and quantification of Her2-targeted gold nanoparticles within tumour regions. Br J Radiol 2011, 84(1002): 526-533.
4. Dorsey J F, Sun L, Joh D Y, Witztum A, Kao G D, Alonso-Basanta M, et al. Gold nanoparticles in radiation research: potential applications for imaging and radiosensitization. Transl Cancer Res 2013, 2(4): 280-291.
5. Taupin F, Flaender M, Delorme R, Brochard T, Mayol J F, Arnaud J, et al. Gadolinium nanoparticles and contrast agent as radiation sensitizers. Phys Med Biol 2015, 60(11): 4449-4464.
6. McQuaid H N, Muir M F, Taggart L E, McMahon S J, Coulter J A, Hyland W B, et al. Imaging and radiation effects of gold nanoparticles in tumour cells. Sci Rep 2016, 6: 19442.
7. Zhu J, Zhao L, Cheng Y, Xiong Z, Tang Y, Shen M, et al. Radionuclide (131)1-labeled multifunctional dendrimers for targeted SPECT imaging and radiotherapy of tumors. Nanoscale 2015, 7(43): 18169-18178.
8. Mi Y, Shao Z, Vang J, Kaidar-Person O, Wang A Z. Application of nanotechnology to cancer radiotherapy. Cancer Nanotechnol 2016, 7(1): 11.
9. Le Duc G, Miladi I, Alric C, Mowat P, Brauer-Krisch E, Bouchet A, et al. Toward an image-guided microbeam radiation therapy using gadolinium-based nanoparticles. ACS Nano 2011, 5(12): 9566-9574.
10. Dufort S, Bianchi A, Henry M, Lux F, Le Duc G, Josserand V, et al. Nebulized gadolinium-based nanoparticles: a theranostic approach for lung tumor imaging and radiosensitization. Small 2015, 11(2): 215-221.
11. Hainfeld J F, Slatkin D N, Smilowitz H M. The use of gold nanoparticles to enhance radiotherapy in mice. Phys Med Biol 2004, 49(18): N309-315.

1. A method of treating a tumor by ionizing radiations in a subject in need thereof, the method comprising
(i) injecting a first therapeutically effective amount of high-Z element containing nanoparticles as radiosensitizing agents in said subject in need thereof within a period between 2 and 10 days, preferably 2 and 7 days, prior to the first irradiation of the tumor,
(ii) injecting a second therapeutically effective amount of the same or different high-Z element containing nanoparticles within a period between 1 hour to 12 hours prior to the first irradiation of the tumor, and,
(iii) irradiating the tumor of said subject with a therapeutically efficient dose of radiations;
wherein said high-Z element containing nanoparticles are nanoparticles containing an element with an atomic Z number higher than 40, preferably higher than 50, and said nanoparticles have a mean hydrodynamic diameter below 10 nm, for example between 1 and 8 nm, preferably below 6 nm, more preferably between 2 and 6 nm.

2. The method of claim 1, wherein said nanoparticles are injected intravenously.

3. The method of claim 1, wherein said nanoparticles comprise, as high-Z element, a rare earth metal, or a mixture of rare earth metals.

4. The method of claim 1, wherein said nanoparticles comprise, as high-Z element, gadolinium, bismuth, or a mixture thereof.

5. The method of claim 1, wherein said nanoparticles comprise chelates of high-Z element, for example chelates of rare earth elements.

6. The method of claim 1, wherein said nanoparticles comprise
polyorganosiloxane,
chelates covalently bound to said polyorganosiloxane,
high-Z elements complexed by the chelates.

7. The method of claim 1, wherein said nanoparticles comprise
polyorganosiloxane with a silicon weight ratio of at least 8% of the total weight of the nanoparticle, preferably between 8% and 50%,
chelates covalently bound to said polyorganosiloxane, in a proportion comprising between 5 and 100, preferably between 5 and 20 per nanoparticle, and,
high-Z elements complexed to the chelates.

8. The method of claim 1, wherein said nanoparticles comprises chelates for complexing the high-Z elements, obtained by grafting one or more of the following cheating agents on said nanoparticles: DOTA, DTPA, EDTA, EGTA, BAPTA, NOTA, DOTAGA, and DTPABA, or their mixtures.

9. The method of claim 1, wherein said nanoparticles are gadolinium-chelated polysiloxane nanoparticles of the following formula 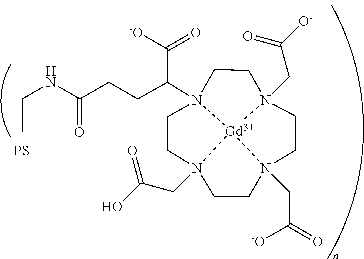
wherein PS is a matrix of polysiloxane, and,
n is comprised between 5 and 50, preferably 5 and 20, and wherein the hydrodynamic diameter is comprised between 2 and 6 nm.

10. The method of claim 1, wherein said nanoparticles are a lyophilized powder contained in a pre-filled vial to be reconstituted in an aqueous solution for intravenous injection.

11. The method of claim 1, wherein said nanoparticles are comprised in an injectable solution at a concentration between 50 and 150 mg/mL, and preferably between 80 and 120 mg/mL, for example 100 mg/mL.

12. The method of claim 1, wherein a therapeutically effective amount administered at each injecting step is comprised between 50 mg/kg and 150 mg/kg, typically, between 80 and 120 mg/kg, for example 100 mg/kg.

13. The method of claim 1, wherein said tumor is a solid tumor, preferably selected from the group consisting of glioblastoma, brain metastases, meniningioma, or primary tumor of uterine cervix, rectum, lung, head and neck, prostate, colorectal, liver, and pancreas cancers.

14. The method of claim 1, wherein said tumor is a brain metastase, typically a brain metastase from melanoma, lung, breast, kidney primary cancers.

15. The method of claim 14, wherein said subject is exposed at step (iii) to a whole brain radiation therapy.

16. The method of claim 15, wherein said whole brain radiation therapy consists of exposing the subject to a total dose of ionizing radiations between 25 and 35 Gy, for example 30 Gy.

17. The method of claim 15, wherein the subject is exposed to a dose of ionizing radiations per fraction of about 3 Gy, and the total dose is administered preferably in a maximum of 10 fractions.

18. The method of claim 1, wherein the method comprises a step of injecting a third therapeutically effective amount of the same or different high-Z element containing nanoparticles within 5-10 days after the second injecting step, for example 7 days after the second injecting step.

19. The method of claim 1, wherein the method further comprises a step of imaging the tumor by magnetic resonance imaging (MRI) after the first injecting step of said nanoparticles, wherein said nanoparticles is used as a T1 contrast agent for said MRI.

20. High-Z element containing nanoparticles, for use in a method of treating a tumor by ionizing radiations in a subject in need thereof, the method comprising
(i) injecting a first therapeutically effective amount of high-Z element containing nanoparticles as radiosensitizing agents in said subject in need thereof within a period between 2 and 10 days, preferably 2 and 7 days, prior to the first irradiation of the tumor,
(ii) injecting a second therapeutically effective amount of the same or different high-Z element containing nanoparticles within a period between 1 hour to 12 hours prior to the first irradiation of the tumor, and,
(iii) irradiating the tumor of said subject with a therapeutically efficient dose of radiations;
wherein said high-Z element containing nanoparticles are nanoparticles containing an element with an atomic Z number higher than 40, preferably higher than 50, and said nanoparticles have a mean hydrodynamic diameter below 10 nm, for example between 1 and 8 nm, preferably below 6 nm, more preferably between 2 and 6 nm.
